(19) United States
(12) Patent Application Publication
(10) Pub. No.: US 2015/0063668 A1
(43) Pub. Date: Mar. 5, 2015

(54) THREE-DIMENSIONLAL VIRTUAL LIVER SURGERY PLANNING SYSTEM

(71) Applicants: POSTECH ACADEMY-INDUSTRY FOUNDATION, Pohang-si (KR); INDUSTRIAL COOPERATION FOUNDATION OF CHONBUK NATIONAL UNIVERSITY, Jeonju-si (KR)

(72) Inventors: Heecheon You, Pohang-si (KR); Xiaopeng Yang, Pohang-si (KR); Younggeun Choi, Pohang-si (KR); Wonsup Lee, Pohang-si (KR); Baik Hwan Cho, Jeonju-si (KR); Hee Chui Yu, Jeonju-si (KR)

(73) Assignees: POSTECH ACADEMY-INDUSTRY FOUNDATION, Pohang-si (KR); INDUSTRIAL COOPERATION FOUNDATION OF CHONBUK NATIONAL UNIVERSITY, Jeonju-si (KR)

(21) Appl. No.: 14/381,318

(22) PCT Filed: Mar. 4, 2013
(86) PCT No.: PCT/KR2013/001717

§ 371 Date: Aug. 27, 2014

(30) Foreign Application Priority Data
Mar. 2, 2012 (KR) 10-2012-0021850
Mar. 4, 2013 (KR) 10-2013-0022881

(51) Int. Cl. A61B 19/00; G06F 3/0484; G06T 7/00

(52) U.S. Cl. CPC A61B 19/50; G06T 7/0012; G06T 7/0081; G06T 7/0089; G06F 3/0484; A61B 2019/501; G06T 2200/24; G06T 2207/10081; G06T 2207/20141; G06T 2207/20148; G06T 2200/04; G06T 2207/30056
USPC 382/131

(57) ABSTRACT

An exemplary embodiment of the present invention provides a three-dimensional virtual liver surgery planning system including: a digital imaging and communications in medicine (DICOM) receiving module which receives an abdomen computer tomography (CT) volume data set from a picture archiving and communication system (PACS) server; a DICOM loading and noise removing module which loads the received abdomen CT volume data set and remove noises; a standard liver volume estimation module which estimates a standard liver volume (SLV) from the denoised abdomen CT volume data set; a liver extraction module which extracts a three-dimensional liver region; a vessel extraction module which extracts a three-dimensional vessel region including a portal vein, a hepatic artery, a hepatic vein, and an inferior vena cava (IVC); a tumor extraction module which extracts a three-dimensional tumor region; a liver segmentation module which divides the extracted three-dimensional liver region into several segments using landmarks which are selected by a user or a segmentation sphere; and a liver surgery planning module which makes a three-dimensional liver surgery plan using a resection surface, a liver segments, or the segmentation sphere.

### TECHNICAL FIELD

The present invention relates to a three-dimensional virtual liver surgery planning system.

### BACKGROUND ART

Safety of a major liver resection may be predicted by a relative residual liver volume (% RLV), the ratio of residual to total functional liver volume (TFLV, Total Liver Volume−tumor volume).

For example, Schindl et al. (2005. M. J. Schindl, D. N. Redhead, K. C. H. Fearon, O. J. Garden, and S. J. Wigmore; “The Value of Residual Liver Volume as a Predictor of Hepatic Dysfunction and Infection After Major Liver Resection,” Gut 54(2), 289-296.) have reported that when % RLV of 104 patients having normal liver function is lower than 27%, an incidence rate of serious hepatic dysfunction after surgery is 90% or higher, and when % RLV is 27% or higher, the incidence rate of hepatic dysfunction is 13%. Ferrero et al. (2007. A. Ferrero, L. Vigano, R. Polastri, A. Muratore, H. Eminefendic, D. Regge, and L. Capussotti; “Postoperative Liver Dysfunction and Future Remnant Liver: Where is the Limit? Results of a Prospective Study;” World Journal of Surgery 31(8), 1643-1651.) suggested that the liver resection is safe when % RLV of a patient having a healthy liver is 26.5% or higher and % RLV of a patient having impaired liver function is 31% or higher based on an analysis result of 119 patients.

A rational liver resection requires appropriate selection of cutting location, orientation and shape of the cutting plane, and is planned by investigating the relative position of a tumor to structures of three liver vessels (portal vein, hepatic vein, and hepatic artery). For safe and rational liver surgery, a three-dimensional virtual liver surgery planning system needs to provide not only visual information such as the position and size of a tumor, structures of hepatic vessels, and liver segments, but also quantitative information such as volumes of the liver, the residual liver (remnant), and the liver to be transplanted (graft).

Most existing virtual surgery systems such as Rapidia (Infinitt Co., Ltd., South Korea), Voxar 3D (Toshiba Co., Japan), Syngovia (Siemens Co., Germany), and OsriX (Pixmeo Co., Switzerland) do not provide a function specialized to liver surgery planning. Further, such a general virtual surgery system provides an insufficient function to be clinically utilized so that surgeons can plan the liver surgery before performing the surgery. For example, a manual or semi-auto liver extraction function which is provided by a general virtual surgery system requires a long processing time (30 minutes or longer) and significant effort by users. Furthermore, the general virtual operation system does not provide functions for liver segments identification and liver surgery planning.

### DISCLOSURE

### Technical Problem

The present invention has been made in an effort to provide a three-dimensional virtual liver surgery planning system which provides 1) a procedure-based user interface, 2) three-dimensional CT image processing, 3) volume calculation, 4) two-dimensional and three-dimensional contour editing, 5) a visualizing technique, and 6) a manipulation technique for safe and rational liver surgery planning.

An exemplary embodiment of the present invention provides a three-dimensional virtual liver surgery planning system including: a digital imaging and communications in medicine (DICOM) receiving module which receives an abdomen computer tomography (CT) volume data set from a picture archiving and communication system (PACS) server; a DICOM loading and noise removing module which loads the received abdomen CT volume data set and remove noises; a standard liver volume estimation module which estimates a standard liver volume (SLV) from the denoised abdomen CT volume data set; a liver extraction module which is connected to the standard liver volume estimation module to extract a three-dimensional liver region; a vessel extraction module which is connected to the liver extraction module to extract a three-dimensional vessel region including a portal vein, a hepatic artery, a hepatic vein, and an inferior vena cava (IVC); a tumor extraction module which is connected to the vessel extraction module to extract a three-dimensional tumor region; a liver segmentation module which divides the extracted three-dimensional liver region into several segments using landmarks which are selected by a user or a segmentation sphere; and a liver surgery planning module which is connected to the liver segmentation module to make a three-dimensional liver surgery plan using a resection surface, liver segments, or the segmentation sphere.

The system may further include a procedure-based user-friendly interface which is connected to the modules.

The system may further include a liver extraction correction module which interacts with the liver extraction module and edits the extracted three-dimensional liver region.

The system may further include a vessel extraction correcting module which interacts with the vessel extraction module and edits the extracted three-dimensional vessel region.

The system may further include a tumor extraction correction module which interacts with the tumor extraction module and edits the extracted three-dimensional tumor region.

The system may further include a liver segmentation correction module which interacts with the liver segmentation module and edits the divided liver segments.

The liver extraction module may perform a semi-automatic hybrid liver extraction method.

The semi-automatic hybrid liver extraction method may include: deriving an initial liver region by applying a fast-marching level set method using multiple seed points selected from a plurality of CT slices by a user; improving the derived initial liver region by a threshold-based level set method; and correcting the extracted liver region using scalable circle in a two-dimensional point of view or using scalable ball in a three-dimensional point of view.

In the correcting, the three-dimensional point of view may be selected using a combined three-dimensional point of view resetting button in which buttons at a front side, a rear side, a left side, a right side, an upper side, and a lower side are combined.

The vessel extraction module may extract three-dimensional regions of the portal vein, the hepatic artery, and the hepatic vein using a region growing method which uses a threshold interval and a seed point.

The region growing method may include: loading a CT volume overlaid with the extracted liver region; editing the liver region using the segmentation sphere so as to include a vessel to be extracted; masking the CT volume with the edited liver region; inputting multiple seed points selected by the user to a plurality of CT slices; searching an initial threshold interval in accordance with an intensity distribution of a data set of the masked CT volume; creating several additional threshold intervals by adjusting a lower threshold and an upper threshold of the initial threshold interval; and extracting multiple vasculatures based on the initial threshold interval and additional threshold intervals to provide the vasculatures together with the volume information in the three-dimensional point of view.

The region growing method may further include editing the extracted vessel using a circle or a segmentation sphere in a two-dimensional or three-dimensional point of view by a user.

The liver segmentation module may be configured to: form a segment 1; divide the liver into a left lobe and a right lobe; divide the right lobe into an anterior sector and a posterior sector; divide the left lobe into a medial sector (segment 4) and a lateral sector; divide the posterior sector into a segment 6 and a segment 7; divide the anterior sector into a segment 5 and a segment 8; and divide the lateral sector into a segment 2 and a segment 3.

Real-time calculated volume values of the extracted liver, vessels, tumors, and liver segments may be represented.

### Advantageous Effects

According to the three-dimensional virtual liver surgery planning system according to an exemplary embodiment of the present invention, a user may easily extract a liver, vessels, and tumors, divide liver segments, and make a surgery plan using a user-friendly interface in accordance with a procedure.

### DESCRIPTION OF DRAWINGS

FIG. 1 is an entire schematic diagram of a three-dimensional virtual liver surgery planning system according to an exemplary embodiment of the present invention.

FIG. 2 is an information processing flowchart of a liver extraction step of a three-dimensional virtual liver surgery planning system according to an exemplary embodiment of the present invention.

FIG. 3 shows images illustrating an example of selecting a seed point for extracting the liver in the liver extraction step of a three-dimensional virtual liver surgery planning system according to an exemplary embodiment of the present invention.

FIG. 4 shows images illustrating an example in which a region of the liver which is extracted in the liver extraction step of the three-dimensional virtual liver surgery planning system according to an exemplary embodiment of the present invention is edited using a three-dimensional scalablesphere in a three-dimensional point of view.

FIG. 5 is a vessel extraction flowchart in the vessel extraction step of the three-dimensional virtual liver surgery planning system according to the exemplary embodiment of the present invention.

FIGS. 6A to 6F show images illustrating a function of providing six expected vasculature candidates extracted based on various threshold intervals so as to select an optimal extraction result by a user in the three-dimensional virtual liver surgery planning system according to the exemplary embodiment of the present invention, and FIGS. 6G and 6H are a graph and a table, respectively, illustrating volumes of expected vasculature candidates.

FIG. 7 is a view illustrating a combined three-dimensional point of view resetting button having a color scheme in the three-dimensional virtual liver surgery planning system according to the exemplary embodiment of the present invention.

FIG. 8 is a flowchart illustrating a three-step procedure based on a flat resection surface for liver segment classification in the three-dimensional virtual liver surgery planning system according to the exemplary embodiment of the present invention.

FIG. 9 is a flowchart illustrating a three-step procedure based on a segmentation sphere for liver segment classification in the three-dimensional virtual liver surgery planning system according to the exemplary embodiment of the present invention.

FIG. 10 is a flowchart illustrating a liver segments classifying procedure in the three-dimensional virtual liver surgery planning system according to the exemplary embodiment of the present invention.

FIG. 11 shows an image illustrating a landmark used to divide the liver into the right lobe and a left lobe in the three-dimensional virtual liver surgery planning system according to the exemplary embodiment of the present invention.

FIG. 12 shows images illustrating the liver which is divided into the right lobe and left lobe by a segmentation sphere in the three-dimensional virtual liver surgery planning system according to the exemplary embodiment of the present invention.

FIG. 13A shows an image illustrating an example of a surgery plan by selecting liver segments to be resected in the three-dimensional virtual liver surgery planning system according to the exemplary embodiment of the present invention, and FIG. 13B shows an explanatory node illustrating a volume for every segment and a color table.

FIG. 14 shows an image illustrating an example of a surgery plan using a segmentation sphere in the three-dimensional virtual liver surgery planning system according to the exemplary embodiment of the present invention.

FIGS. 15A to 15F show images illustrating an example of a procedure-based user interface in the three-dimensional virtual liver surgery planning system according to the exemplary embodiment of the present invention.

### MODE FOR INVENTION

Hereinafter, the present invention will be described more fully with reference to the accompanying drawings, in which exemplary embodiments of the invention are shown. The drawings and description are to be regarded as illustrative in nature and not restrictive. Like reference numerals designate like elements throughout the specification.

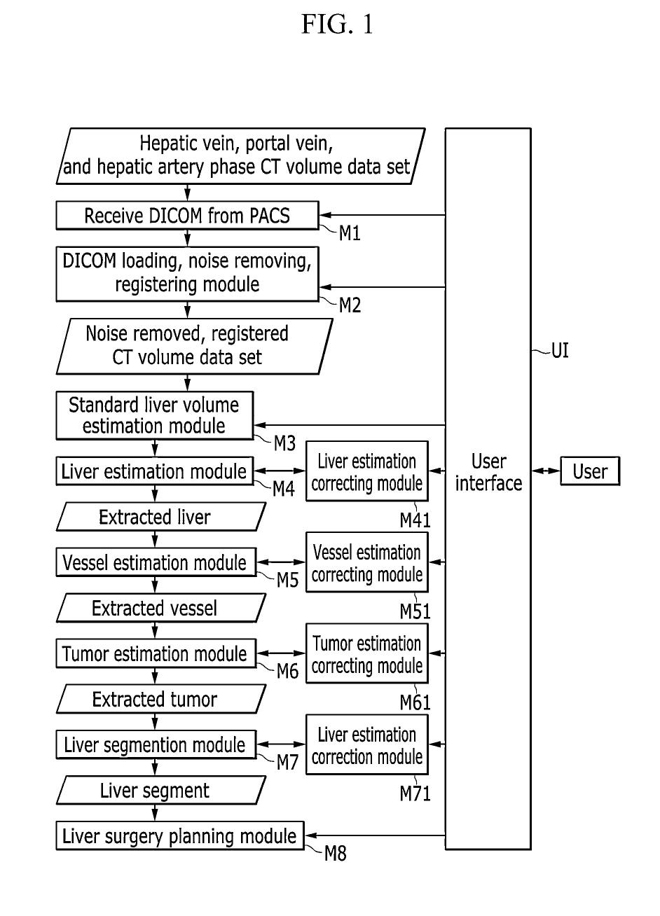

FIG. 1 is an entire schematic diagram of a three-dimensional virtual liver surgery planning system according to an exemplary embodiment of the present invention.

A three-dimensional virtual liver surgery planning system according to an exemplary embodiment of the present invention includes a digital imaging and communications in medicine (DICOM) receiving module M1, a DICOM loading and noise removing module M2, a standard liver volume estimation module M3, a liver extraction module M4, a vessel extraction module M5, a tumor extraction module M6, a liver segmention module M7, and a liver surgery planning module M8, wherein a liver extraction correction module M41 is connected to the liver extraction module M4, a vessel extraction correction module M51 is connected to the vessel extraction module M5, a tumor extraction correction module M61 is connected to the tumor extraction module M6, and a liver segmention correction module M71 is connected to the liver segmention module M7.

In the system according to the present exemplary embodiment, first, a computer tomography (CT) volume data set from a hepatic artery phase, a portal vein phase, and a hepatic vein phase is input to the system. The DICOM receiving module M1 receives CT volume data of various phases from a picture archiving and communication system (PACS) server and stores the CT volume data in a local storage. The DICOM loading and noise removing module M2 loads a CT volume data set in the system to remove a noise and registers the CT volume data set of each phase. The standard liver volume (SLV) estimation module M3 may provide a standard liver volume (SLV) estimated through three regression models.

The liver extraction module M4 uses the CT volume data set of a portal vein phase from which noises are removed in order to extract a liver region. The extracted liver may be visualized as a two-dimensional point of view and a three-dimensional point of view on a CT image, and a user may freely edit the three-dimension liver region from the two-dimensional and three-dimensional points of view using the liver extraction correction module M41. The liver extraction module M4 further provides a function of calculating a volume of the extracted liver.

The vessel extraction module M5 uses a CT volume data set of a hepatic artery phase, a portal vein phase, and a hepatic vein phase which are registered after removing noises as input in order to extract four three-dimensional vessels (the portal vein, the hepatic artery, the hepatic vein, and inferior vena cava). The extracted vessel may be visualized as a two-dimensional point of view and a three-dimensional point of view on the CT image, and a region of the extracted vessel may be efficiently edited by the vessel extraction correction module M51 at the two-dimensional point of view and the three-dimensional point of view. The vessel extraction module M5 further provides a function of calculating a volume of the extracted vessel.

The system according to the present exemplary embodiment provides the tumor extraction module M6 in order to extract tumors from the CT volume data set. The extracted tumor may be visualized as a two-dimensional point of view and a three-dimensional point of view on the CT image, and the user may efficiently edit a region of the extracted tumor using the tumor extraction correction module M61 in both the two-dimensional point of view and the three-dimensional point of view. The tumor extraction module M6 further provides a function of calculating a volume of the extracted tumor.

The system according to the present exemplary embodiment provides the liver segmentation module M7 in order to classify the extracted liver into several segments. The liver segmenting module M7 provides an interactive method in order for the user to select multiple landmarks from the two-dimensional point of view on the CT image, and the selected landmarks may be visualized in the two-dimensional point of view and the three-dimensional point of view. The liver may be divided into several segments based on the resection surface which is generated by the selected landmarks or a segmentation sphere. The divided liver segments may be visualized in the three-dimensional point of view, and a color scheme which may visualize the divided liver with different colors for every segment is also provided. The liver segmentation module M7 provides an interactive method which may adjust a color and transparency of each segment, and also provides a function of calculating the volume of each segment. The liver segmentation may be corrected by the liver segmentation correction module M71.

Finally, the liver surgery planning module M8 uses results from previous modules and provides information on a liver, a vessel, a tumor, a liver segment, and a relative spatial relationship in the liver section, and visualizes the information from the three-dimensional point of view. The surgery planning module M8 provides at least one resection surface in order to cut a part of the liver having a tumor, and provides a function of adjusting a direction, a position, and a shape of the resection surface and cut liver segments having a tumor using the resection surface or a segmentation sphere. Further, the liver surgery planning module M8 visualizes relative liver volume information for an effective surgery plan in a three-dimensional point of view. The user may utilize the liver surgery planning module M8 with a user interface UI in order to perform a virtual liver surgery planning procedure.

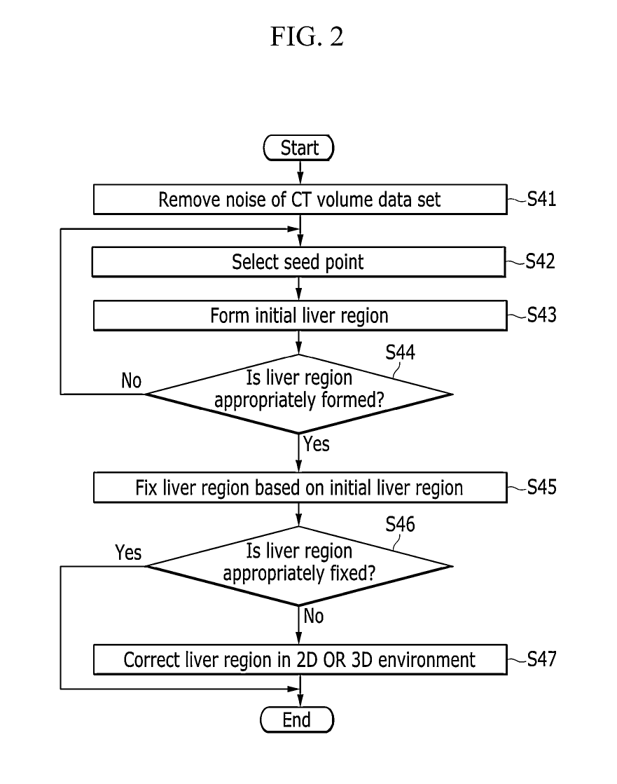

FIG. 2 is an information processing flowchart of a liver extraction step of a three-dimensional virtual liver surgery planning system according to an exemplary embodiment of the present invention. The liver extraction module may perform a semiautomatic hybrid liver extraction method. That is, the liver extraction module automatically extracts an initial liver region based on the selected seed point and then manually extracts a three-dimensional liver region through additional correction. Detailed description thereof will be given below.

First, noises of a CT volume data set of a portal vein phase are removed in step S41.

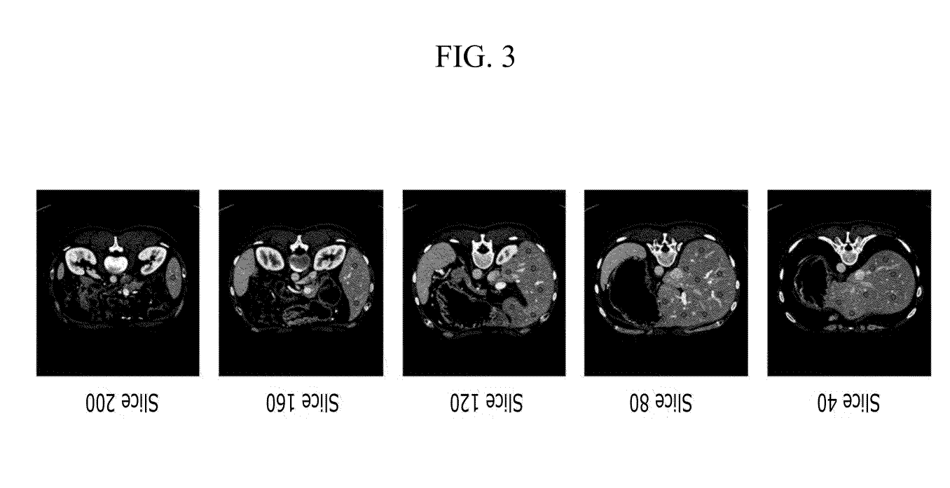

Second, a user selects multiple seed points in a liver region using a mouse in step S42. The selected seed points may be marked as red points, for example. It is desirable to select approximately five slices at the same interval in one CT volume data set. When the liver region is large, 7 to 15 seed points need to be selected for one slice, and when the liver region is small, one to four seed points may be selected for one slice (see FIG. 3). The seed points may be selected so as to embrace the entire liver region including an edge.

Third, an initial liver region is derived by a fast marching level-set method in step S43. When the derived initial liver region is inappropriately too large, the user may return to the seed point selection step to select seed points again.

Fourth, the liver region which is initially formed may be extended by a threshold-based level-set method in step S45. If the extracted liver region is appropriate, the liver extraction process may be finished in step S46. However, when the user wants to edit the extracted liver, the user may remove a portion which does not belong to the liver or add a missing portion using a scalable circle or sphere in the two-dimensional and three-dimension points of view in step S47. That is, a two-dimensional scalable circle or a three-dimensional scalable sphere is provided so that the user clicks and drags the region which is not the liver to effectively remove the region.

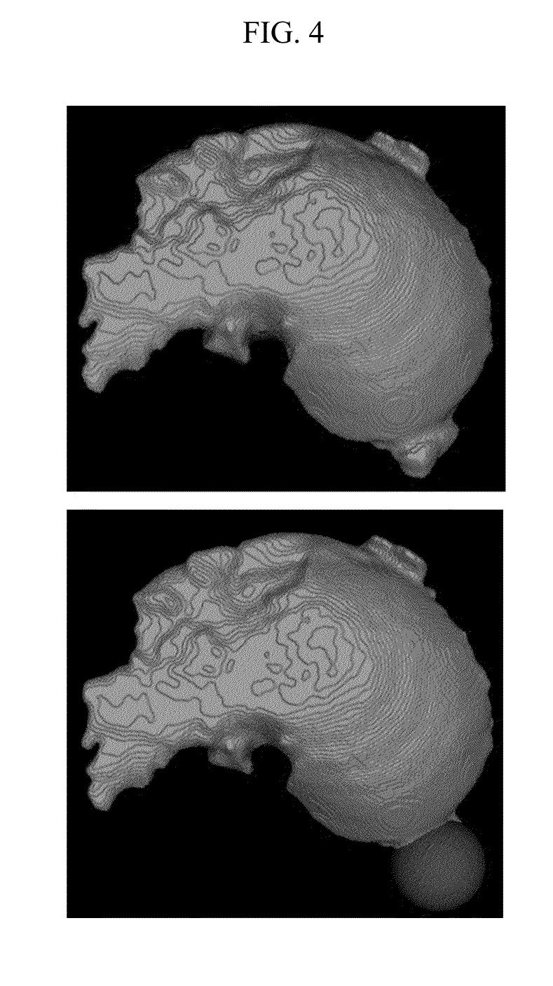

FIG. 4 shows an image illustrating an example of removing a portion which does not belong to the liver region from the extracted liver region using a three-dimensional scalable sphere at a three-dimensional point of view.

Next, a step of smoothing a surface of the corrected liver region may be performed and a volume of the liver extracted in the liver extraction step is calculated to be displayed on a screen.

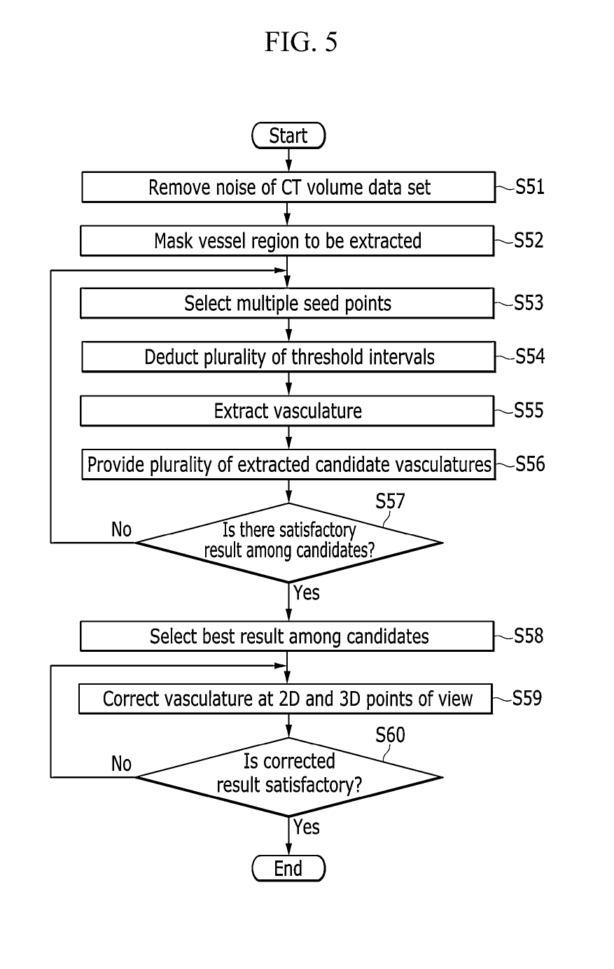

FIG. 5 is a vessel extraction flowchart in the vessel extraction step of the three-dimensional virtual liver surgery planning system according to the exemplary embodiment of the present invention.

First, noises of a CT volume data set of a hepatic artery phase, a portal vein phase, and a hepatic vein phase are removed in step S51.

Second, the user masks a region including a vessel using a masking sphere based on the extracted liver region in step S52.

Third, a user selects multiple seed points in a vessel region of one or two CT slices using a mouse in step S53.

Fourth, an initial threshold interval is automatically derived in accordance with an intensity distribution of the masked CT image, and then five threshold intervals which are different from the derived threshold interval are automatically determined in step S54.

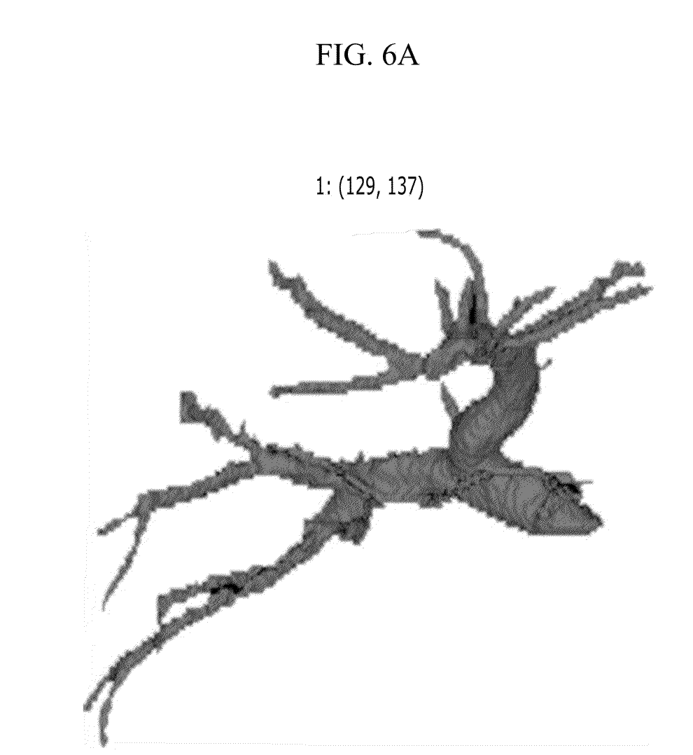

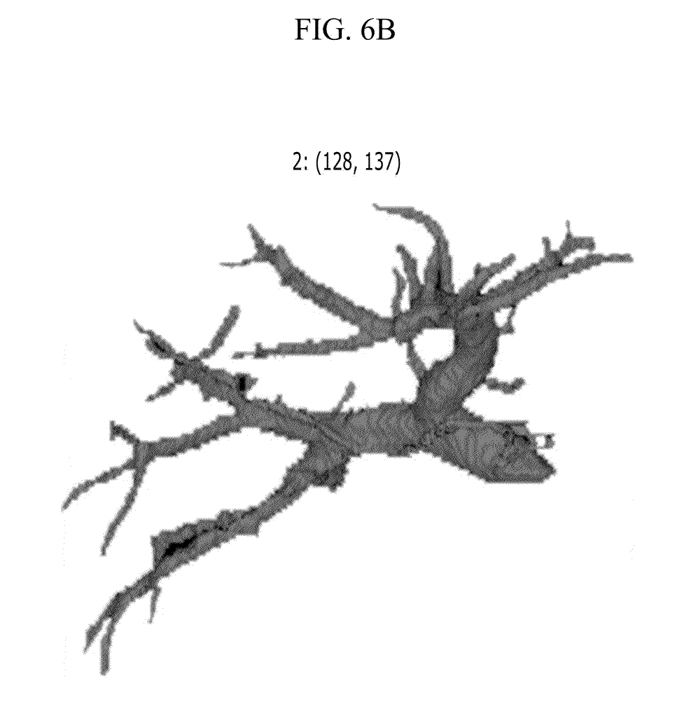

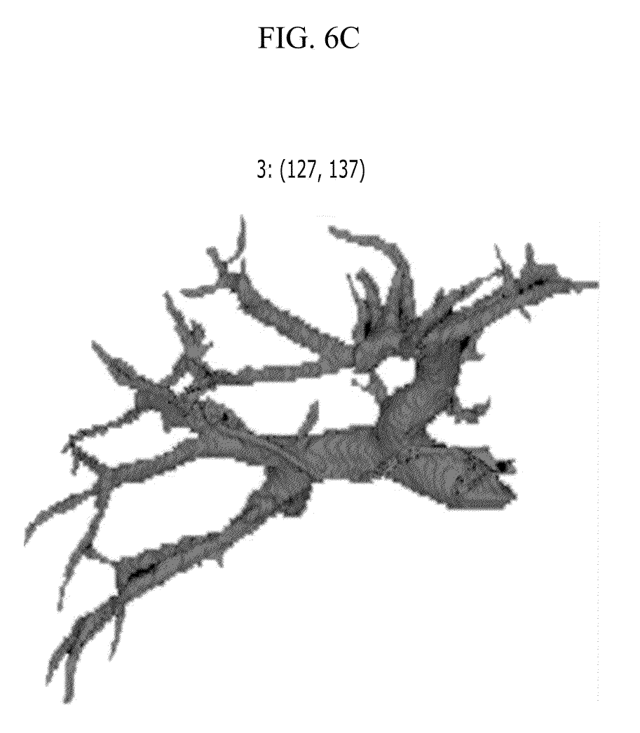

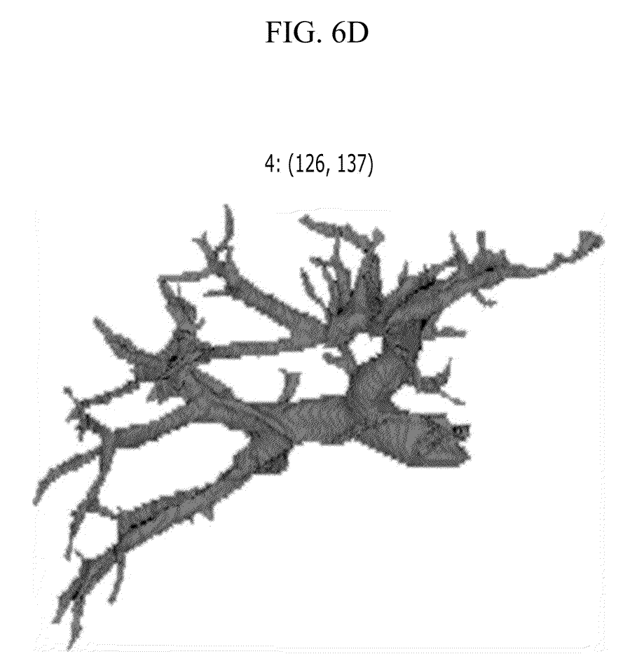

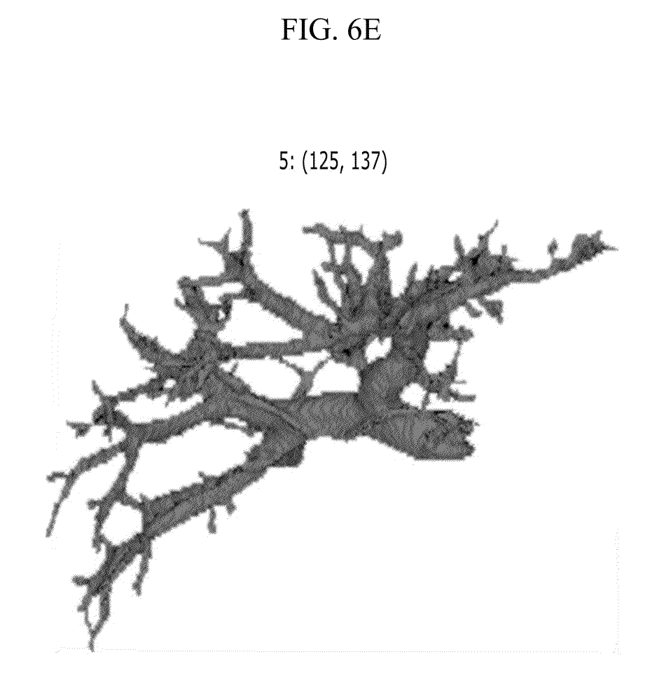

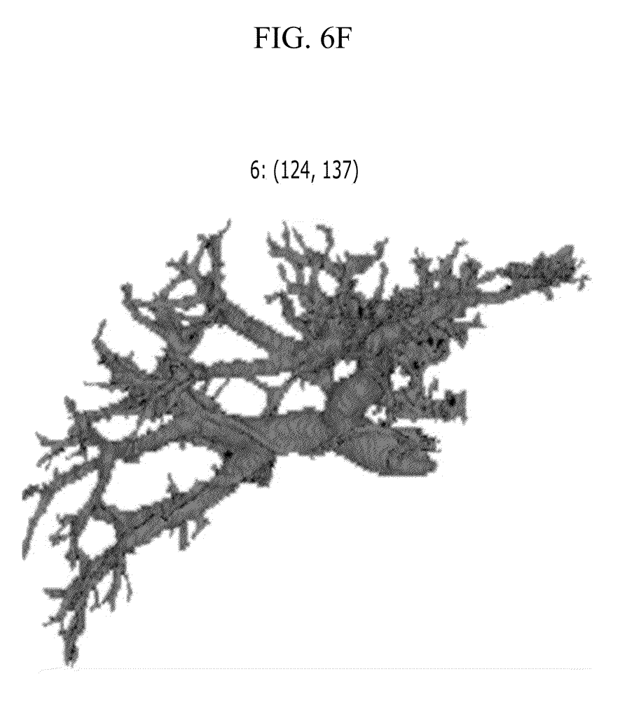

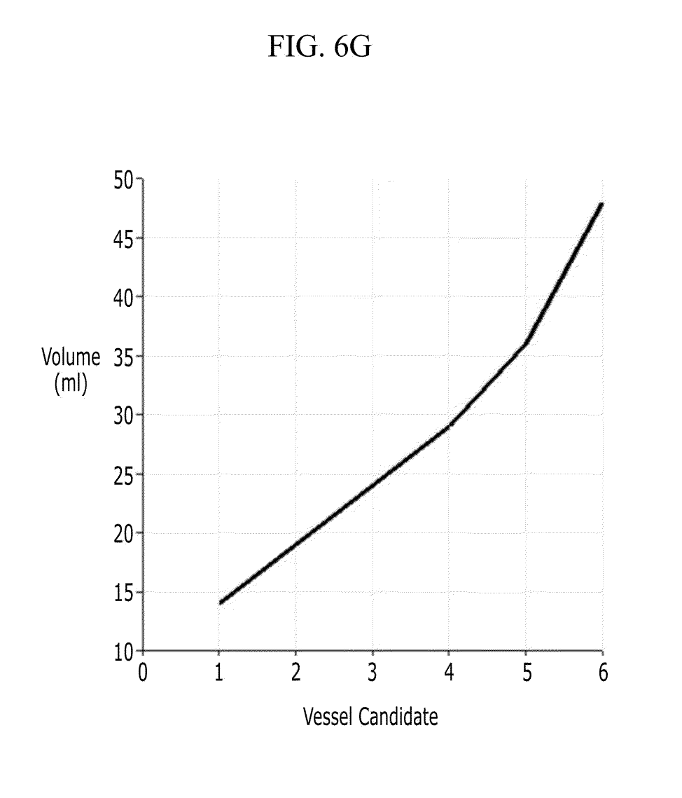

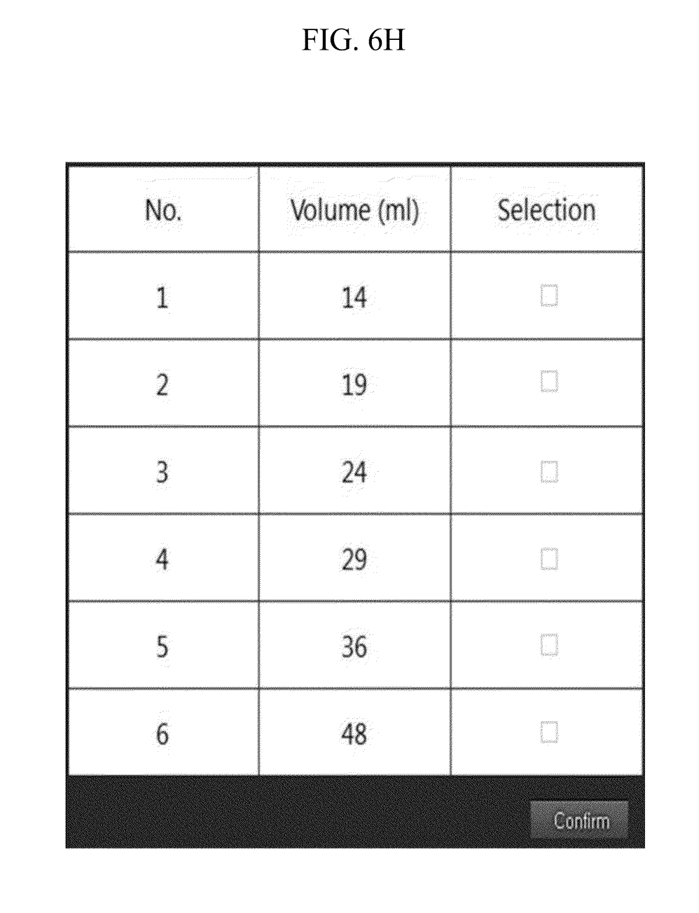

Fifth, a vasculature is extracted by a region growing method based on six threshold intervals in step S55. Next, extracted six vasculature candidates are three-dimensionally shown together with volume information in step S56. It is determined whether a satisfactory result is included in the six vasculature candidates in step S57, and when the satisfactory result is included in the six vasculature candidates, the user may select the result as a final result in step S58 (see FIGS. 6A to 6H). In contrast, when the satisfactory result is not included in the six candidates, the user may repeat the vessel extraction step from the seed point selection step (S53) again.

Finally, the user may correct the selected vasculature from the two-dimensional and three-dimensional points of view in step S59. When the corrected result is satisfactory, the vessel extraction procedure is completed, but when the corrected result is not satisfactory, the correcting step may be performed again.

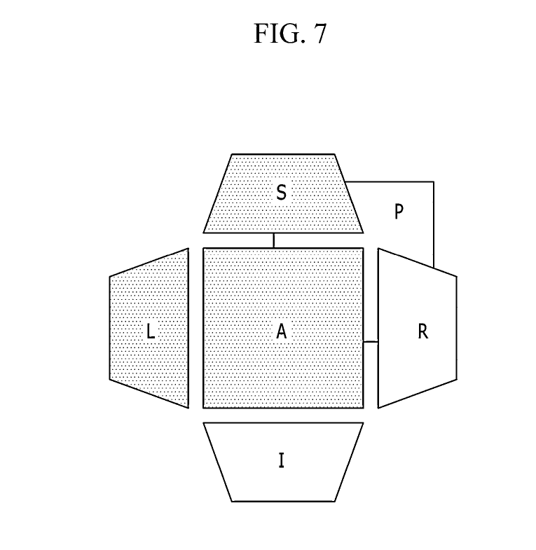

FIG. 7 is a view illustrating combined three-dimensional point of view resetting buttons A, P, S, I, L, and R having a color scheme in the three-dimensional virtual liver surgery planning system according to the exemplary embodiment of the present invention. Two colors are applied to the combined buttons (A, S, and L buttons are sky blue and P, I, and R buttons are yellow). Each button has its own position, and for example, the A button is at a front side, the P button is at a rear side, the L button is at a left side, the R button is at a right side, the S button is at an upper side, and the I button is at a lower side. The user may select any one of combined buttons to reset the three-dimensional point of view to a specific point of view.

That is, when a direction or a position is freely adjusted by the user in a three-dimensional screen, a function of the combined buttons may be set so that the point of view may be restored in a specific direction at one time. For example, when a user wants to immediately change a situation when the extracted three-dimensional liver is watched from the right side to a situation to watch a rear side of the liver, the user presses the P button to change the screen to a point of view in which the liver is watched from the rear side.

Further, in the color scheme, sky blue is assigned to the left, upper, and front sides and yellow is assigned to the right, lower, and rear sides in consideration of a positional meaning of each point of view, which allows a user to easily recognize and utilize the meaning of the button.

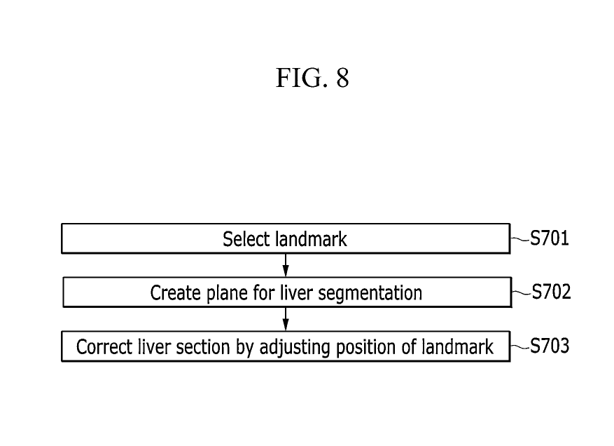

FIG. 8 is a flowchart illustrating a three-step procedure based on a resection surface for liver segment classification in the three-dimensional virtual liver surgery planning system according to the exemplary embodiment of the present invention.

First, the liver may be divided into several sections based on a vasculature of a hepatic vein and a portal vein by the Couinaud model. In order to divide the segments, a user may select landmarks in a two-dimensional CT image using a computer mouse in step S701. The selected points are represented by red color so as to be shown that the selected points are selected as the landmarks. Simultaneously, it may also be displayed using a red sphere at the same position of the three-dimensional point of view where the landmarks are selected.

Second, a resection surface which passes through the selected landmarks is created before segmenting the liver in step S702.

Third, the user adjusts the resection surface to correct the divided liver segments in step S703. A position and an angle of the resection surface may be freely adjusted by dragging in the three-dimensional point of view.

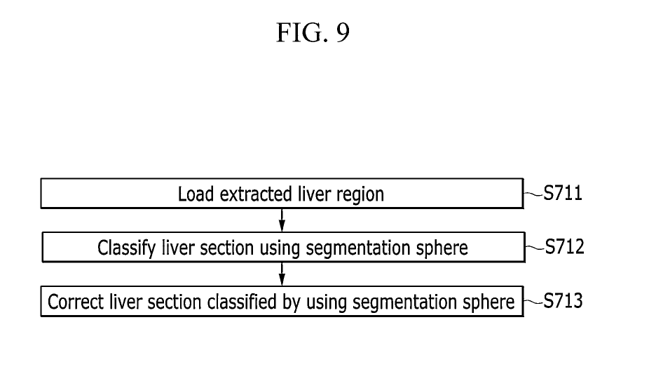

FIG. 9 is a flowchart illustrating a three-step procedure based on a segmentation sphere for liver segment classification in the three-dimensional virtual liver surgery planning system according to the exemplary embodiment of the present invention.

First, an extracted liver region is loaded to overlay an original CT image in step S711.

Next, liver segments in accordance with the Couinaud model are classified through a segmentation sphere in step S712.

Finally, the classified liver segments may be corrected through the segmentation sphere as necessary in step S713.

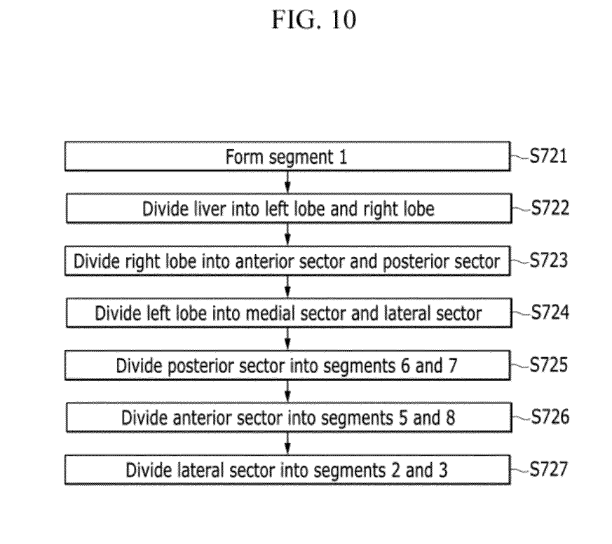

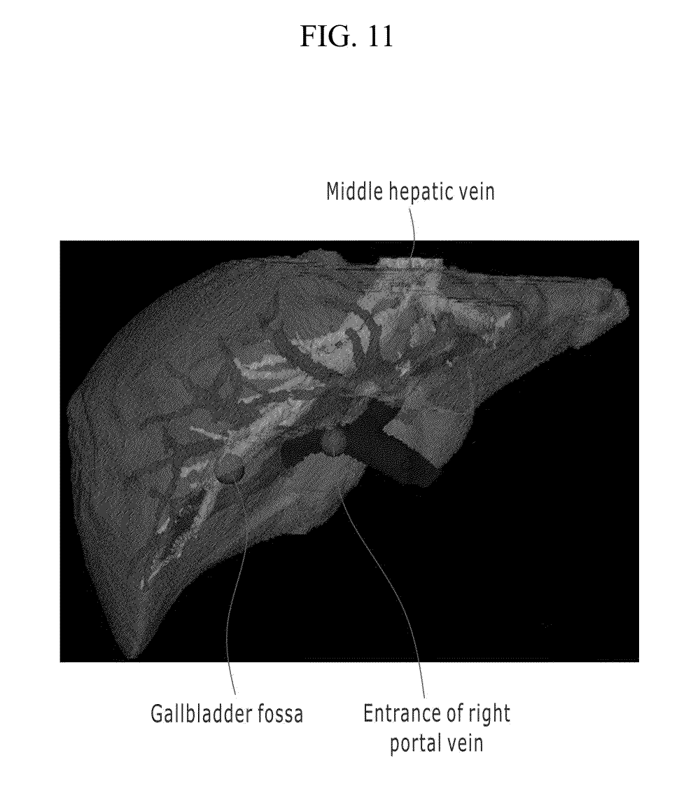

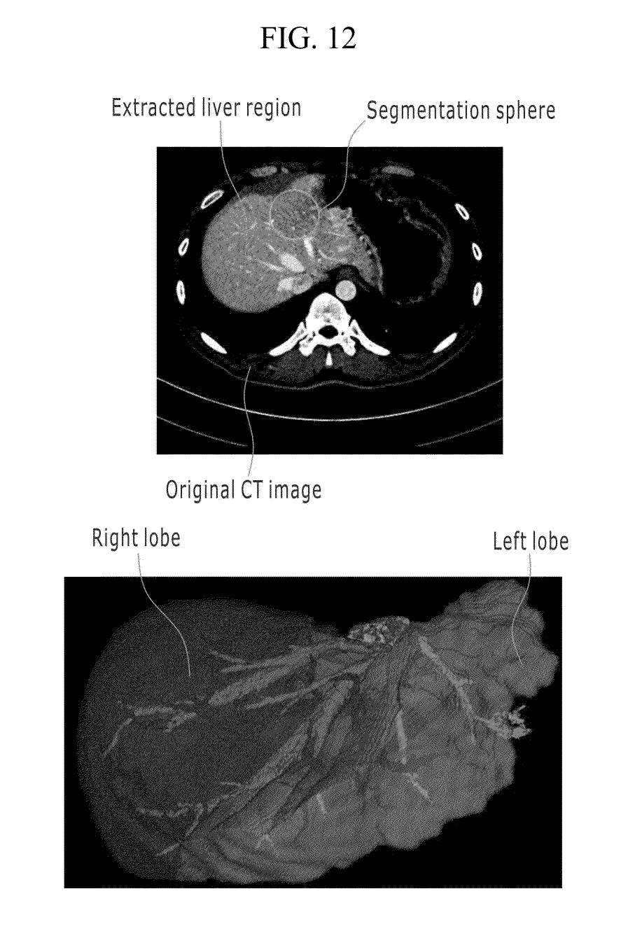

FIG. 10 is a flowchart illustrating a liver segment classifying procedure in the three-dimensional virtual liver surgery planning system according to the exemplary embodiment of the present invention, FIG. 11 shows an image illustrating three landmarks (middle hepatic vein, entrance of right portal vein, and gallbladder fossa) used to divide the liver into a right lobe and a left lobe in the three-dimensional virtual liver surgery planning system according to the exemplary embodiment of the present invention, and FIG. 12 shows images illustrating a three-dimensional liver region which is extracted based a CT image to be divided into a right lobe and a left lobe by a segmentation sphere.

First, a segment 1 is formed in step S721.

Second, the remaining liver is divided into a right lobe and a left lobe by an resection surface which passes the middle hepatic vein, an entrance of the right portal vein, and a gallbladder fossa (see FIG. 11), or the segmentation sphere (see FIG. 12) in step S722.

Third, the right lobe may be divided into an anterior sector and a posterior sector along a right hepatic vein in step S723.

Fourth, the left lobe may be divided into a medial sector (segment 4) and a lateral sector along a left hepatic vein in step S724.

Fifth, the posterior sector may be divided into a segment 6 and a segment 7 in accordance with a right posterior portal vein vasculature in step S725.

Sixth, the anterior sector may be divided into a segment 5 and a segment 8 in accordance with a right anterior portal vein vasculature in step S726.

Finally, the lateral sector may be divided into a segment 2 and a segment 3 in accordance with a left portal vein vasculature in step S727.

In the liver segmentation process, segments other than the segment 1 may be classified based on the vasculature of the portal vein and the hepatic vein by the resection surface created based on the landmarks and the segmentation sphere.

The liver segmentation may be performed entirely or partially as necessary for the user.

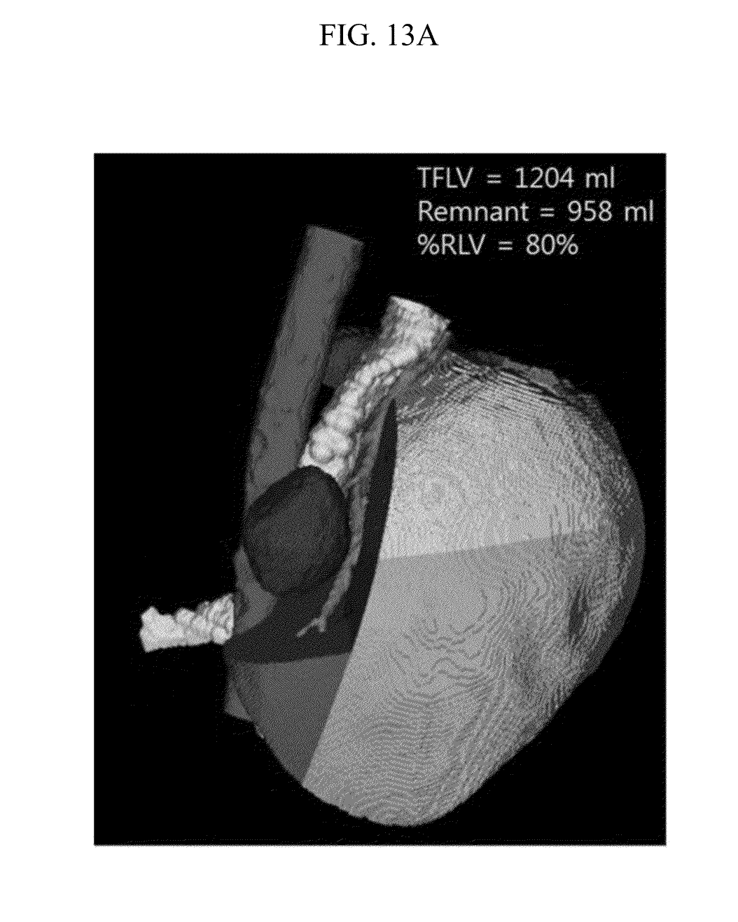

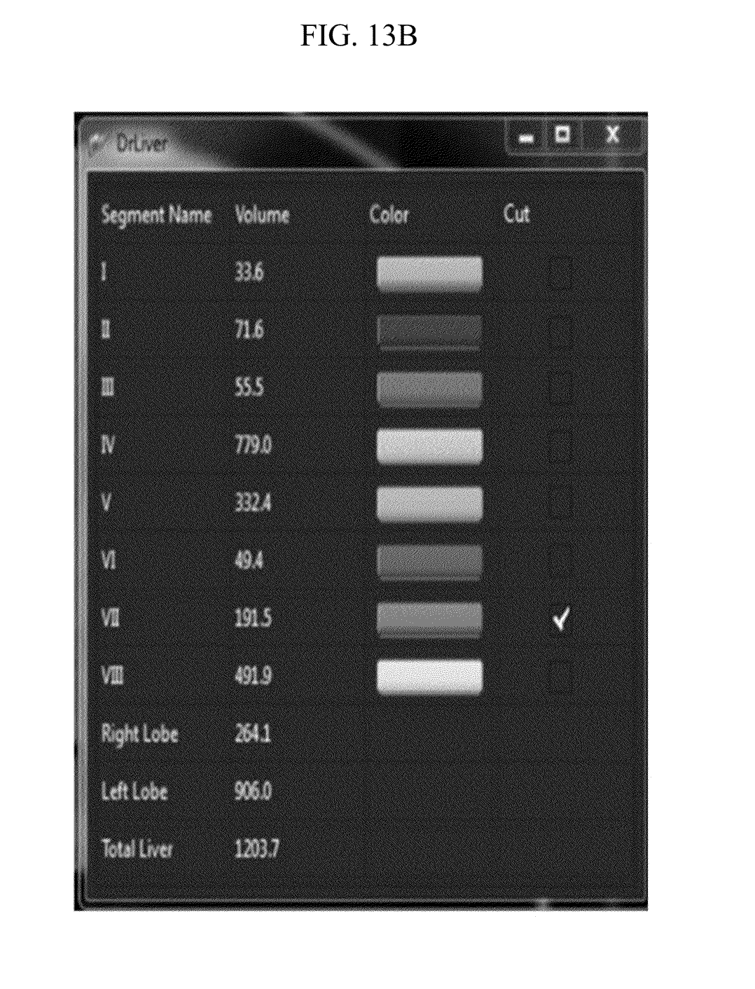

FIG. 13A shows an image illustrating an example of an surgery plan by selecting a liver segment to be resected in the three-dimensional virtual liver surgery planning system according to the exemplary embodiment of the present invention, and FIG. 13B shows an explanatory node illustrating a volume for every segment and a color table.

In this example, a liver segment, a vessel, and a tumor are visualized by a three-dimensional rendering technique. A user may effectively remove a tumor using a function of hiding a segment where a tumor is located. That is, in a liver surgery planning step, a part of a three-dimensional liver where the tumor is located is resected using one or more resection surfaces which are defined by the user or a segmentation sphere, and the three-dimensional liver segments where the tumor is located become completely transparent using a check box to be removed. Further, in order to assist the surgery planning, information on a total liver volume, a volume of a portion to be resected, and a volume and a ratio of a liver which remains after resection may be provided on a three-dimensional screen in real time.

In the liver surgery planning step, a resection surface of the liver has a thickness of 2 mm, which is obtained by a sum of a sectional thickness of a resecting region of 1 mm and a sectional thickness of a region to be resected of 1 mm. That is, in the liver surgery planning step, a predetermined region of a three-dimensional liver is resected. In this case, in order to effectively visualize the resection surface, a three-dimensional object having a thickness is created. In this case, 1 mm is added inside and outside from the resection surface so that the thickness is 2 mm in total.

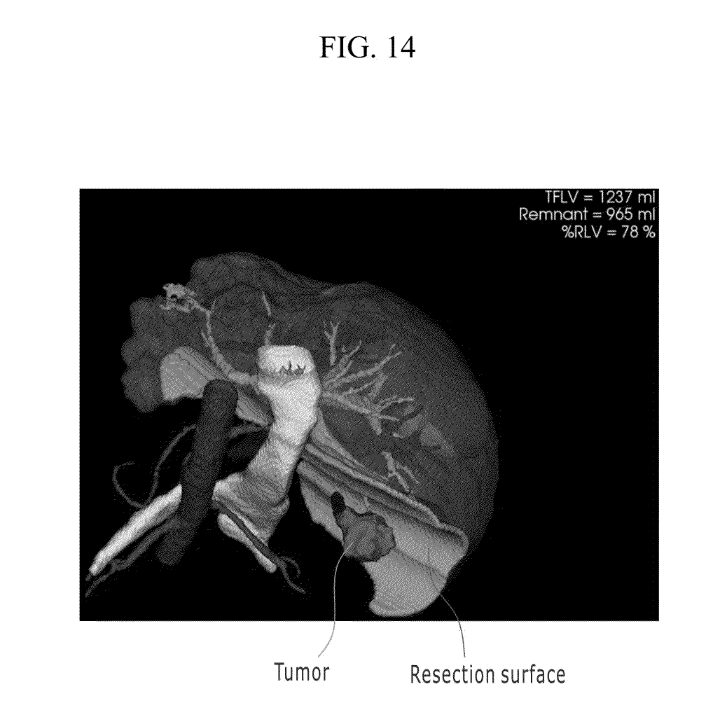

FIG. 14 shows an image illustrating an example of a surgery plan using a segmentation sphere in the three-dimensional virtual liver surgery planning system according to the exemplary embodiment of the present invention.

A user may effectively remove a liver region where a tumor is located using a segmentation sphere. A resection surface of a resected liver is shown in a three-dimensional point of view. Further, volume information such as a total liver volume and a residual liver volume ratio (% RLV) may be provided on the three-dimensional screen in real-time in order to assist a liver section surgery planning.

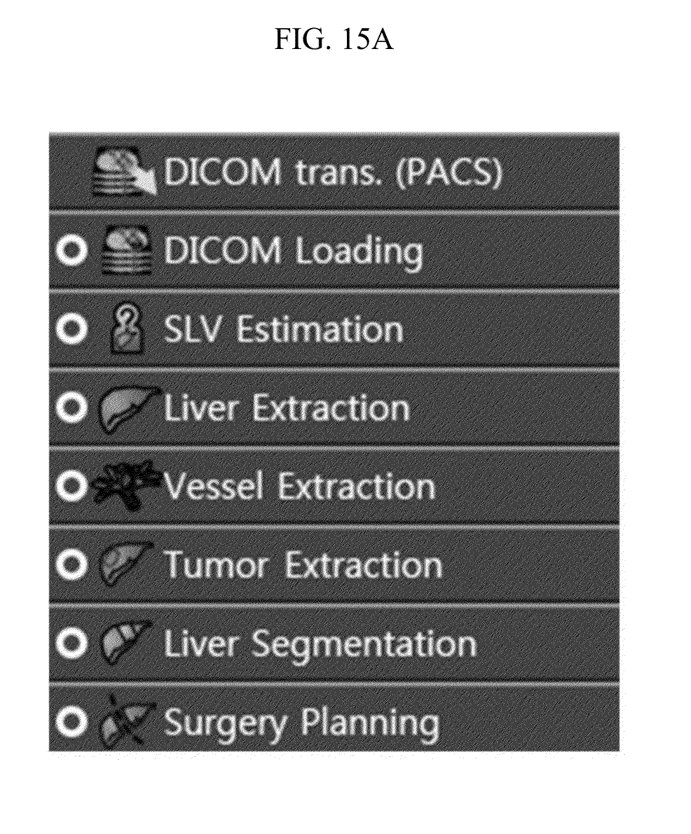

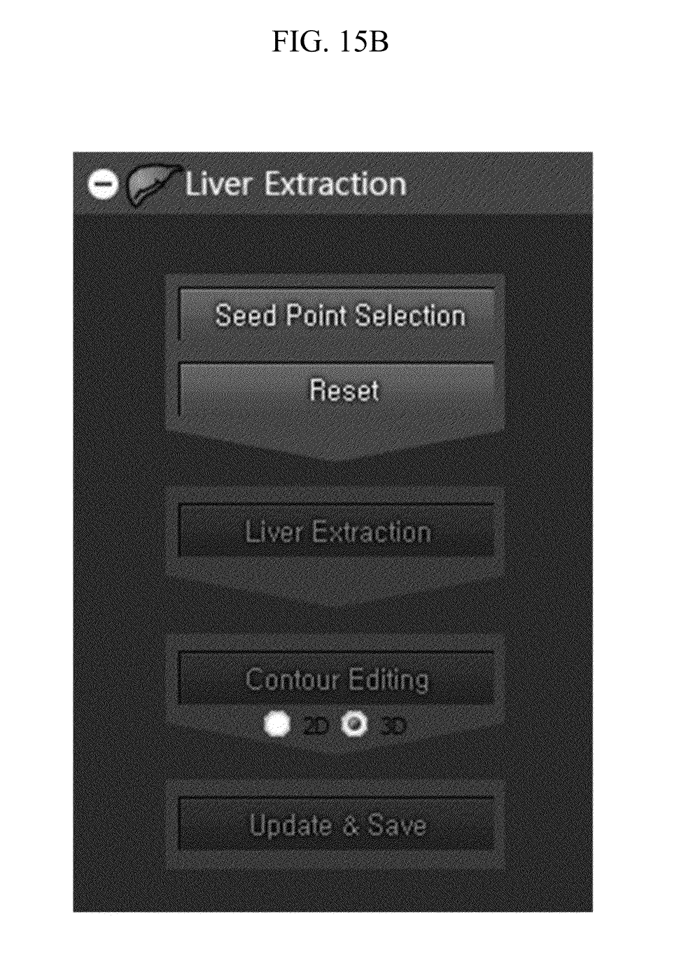

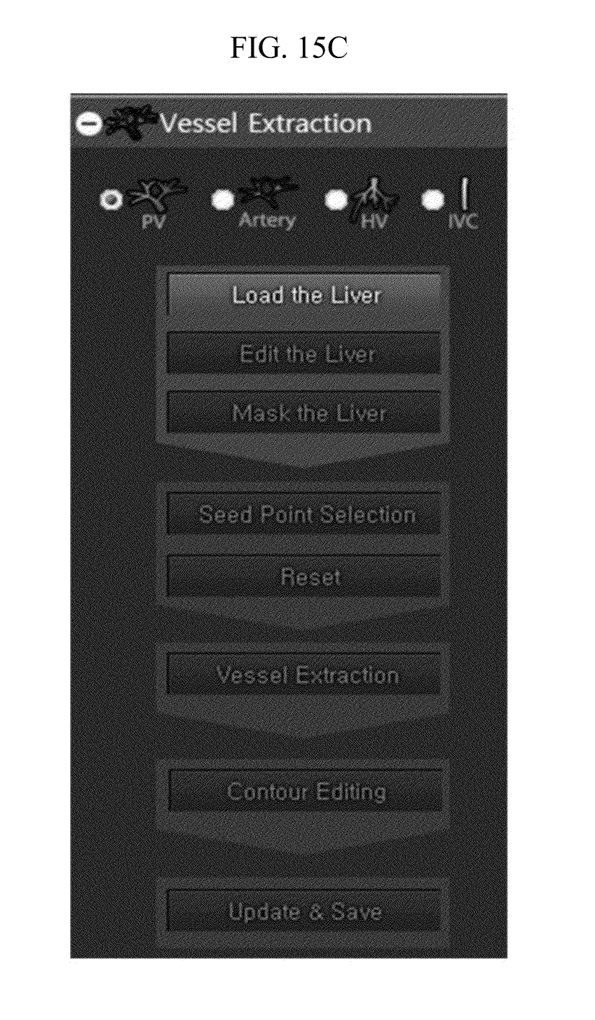

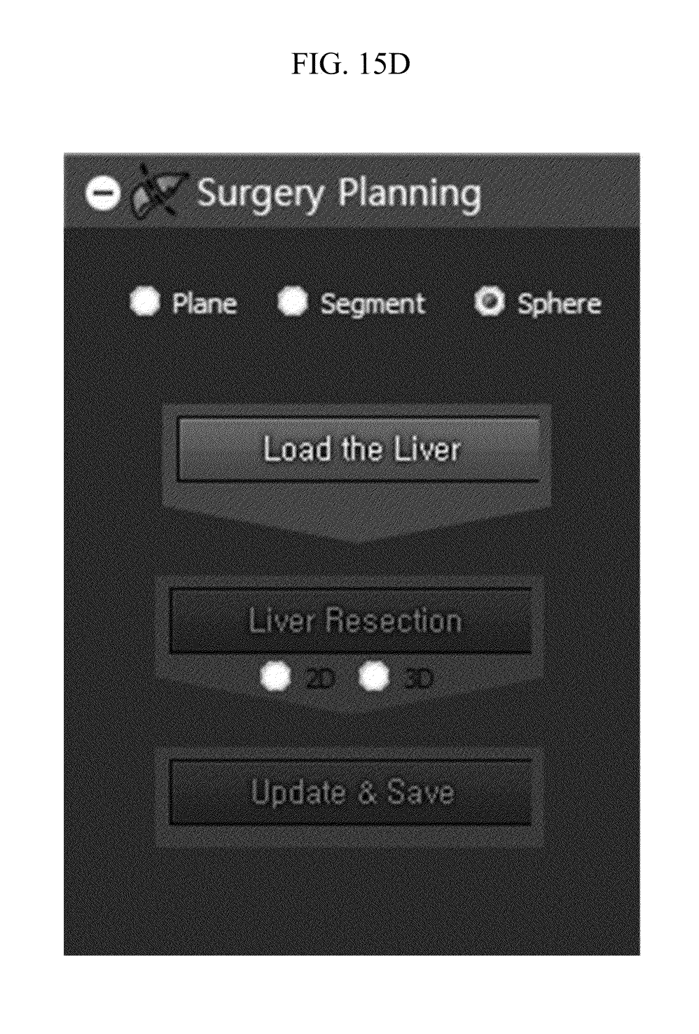

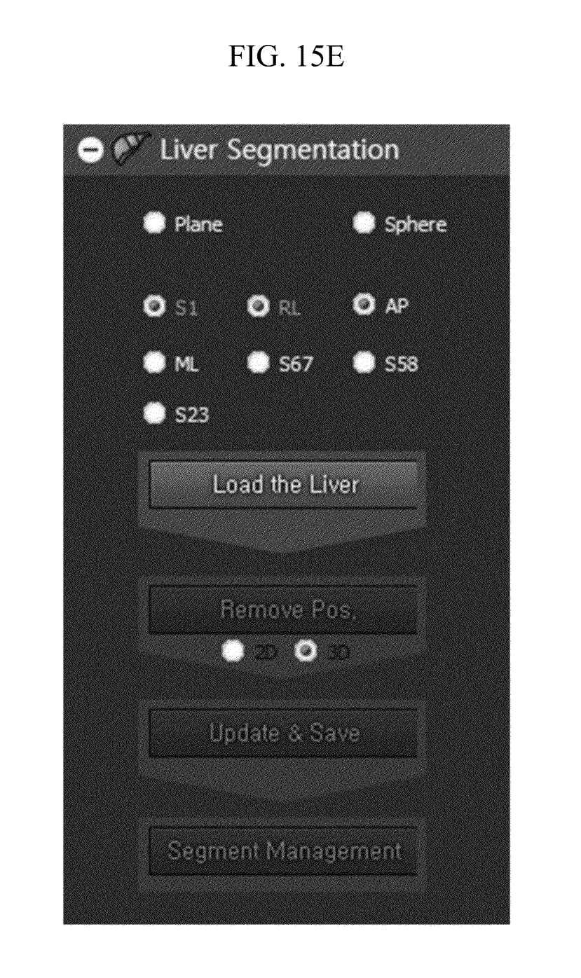

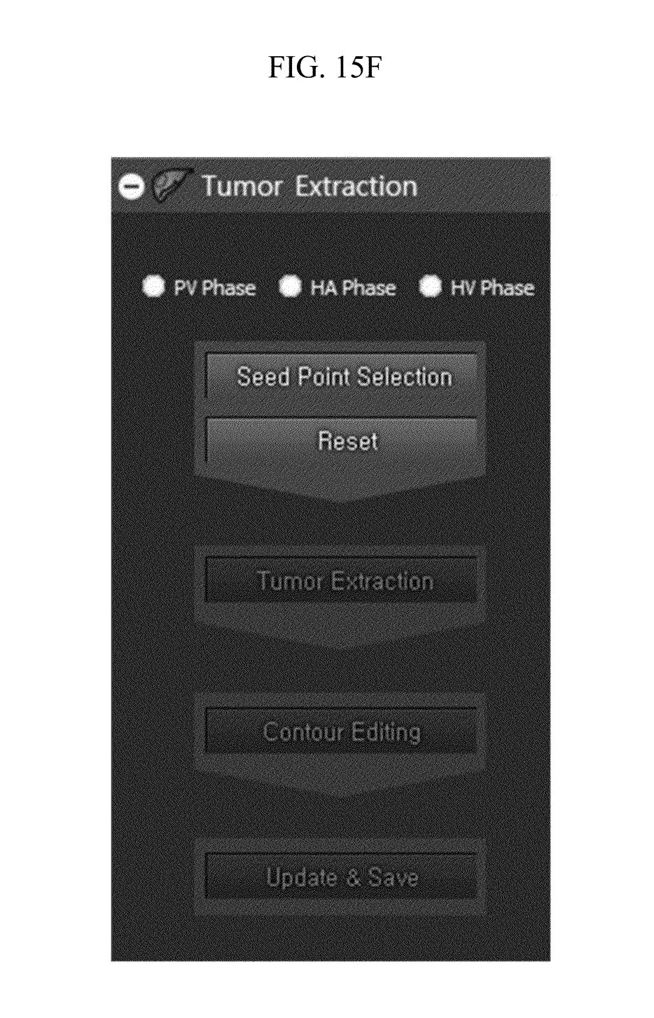

FIGS. 15A to 15F are photographs illustrating an example of a procedure-based user interface in the three-dimensional virtual liver surgery planning system according to the exemplary embodiment of the present invention.

A user may easily extract the liver, vessels, and tumors, divide the liver into different segments, and make a surgery plan using a user-friendly interface in accordance with a procedure.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims.

1. A three-dimensional virtual liver surgery planning system, comprising:
a digital imaging and communications in medicine (DICOM) receiving module which receives an abdomen computer tomography (CT) volume data set from a picture archiving and communication system (PACS) server;
a DICOM loading and noise removing module which loads the received abdomen CT volume data set and remove noises;
a standard liver volume estimation module which estimates a standard liver volume (SLV) from the denoised abdomen CT volume data set;
a liver extraction module which is connected to the standard liver volume estimation module to extract a three-dimensional liver region;
a vessel extraction module which is connected to the liver extraction module to extract a three-dimensional vessel region including a portal vein, a hepatic artery, a hepatic vein, and an inferior vena cava (IVC);
a tumor extraction module which is connected to the vessel extraction module to extract a three-dimensional tumor region;
a liver segmentation module which divides the extracted three-dimensional liver region into a several segments using landmarks which are selected by a user or a segmentation sphere; and
a liver surgery planning module which is connected to the liver segmentation module to make a three-dimensional liver surgery plan using a resection surface, liver segments, or the segmentation sphere.

2. The three-dimensional virtual liver surgery planning system of claim 1, further comprising
a procedure-based user-friendly interface which is connected to the modules.

3. The three-dimensional virtual liver surgery planning system of claim 1, further comprising
a liver extraction correction module which interacts with the liver extraction module and edits the extracted three-dimensional liver region.

4. The three-dimensional virtual liver surgery planning system of claim 1, further comprising
a vessel extraction correcting module which interacts with the vessel extraction module and edits the extracted three-dimensional vessel region.

5. The three-dimensional virtual liver surgery planning system of claim 1, further comprising
a tumor extraction correction module which interacts with the tumor extraction module and edits the extracted three-dimensional tumor region.

6. The three-dimensional virtual liver surgery planning system of claim 1, further comprising
a liver segmentation correction module which interacts with the liver segmentation module and edits the divided liver segments.

7. The three-dimensional virtual liver surgery planning system of claim 1, wherein
the liver extraction module performs a semi-automatic hybrid liver extraction method.

8. The three-dimensional virtual liver surgery planning system of claim 7, wherein
the semi-automatic hybrid liver extraction method includes:
deriving an initial liver region by applying a fast-marching level set method using multiple seed points selected from a plurality of CT slices by a user;
improving the derived initial liver region by a threshold-based level set method; and
correcting the extracted liver region using a scalable circle in a two-dimensional point of view or using a scalable ball in a three-dimensional point of view.

9. The three-dimensional virtual liver surgery planning system of claim 8, wherein,
in the correcting,
the three-dimensional point of view is selected using a combined three-dimensional point of view resetting button in which buttons at a front side, a rear side, a left side, a right side, an upper side, and a lower side are combined.

10. The three-dimensional virtual liver surgery planning system of claim 1, wherein
the vessel extraction module extracts three-dimensional regions of the portal vein, the hepatic artery, and the hepatic vein using a region growing method which uses a threshold interval and a seed point.

11. The three-dimensional virtual liver surgery planning system of claim 10, wherein
the region growing method includes:
loading a CT volume overlaid with the extracted liver region;
editing the liver region using the segmentation sphere so as to include a vessel to be extracted;
masking the CT volume with the edited liver region;
inputting multiple seed points selected by the user to a plurality of CT slices;
searching an initial threshold interval in accordance with an intensity distribution of a data set of the masked CT volume;
creating several additional threshold intervals by adjusting a lower threshold and an upper threshold of the initial threshold interval; and
extracting multiple vasculatures based on the initial threshold interval and additional threshold intervals to provide the vasculatures together with the volume information in the three-dimensional point of view.

12. The three-dimensional virtual liver surgery planning system of claim 11, wherein
the region growing method further includes editing the extracted vessel using a circle or a segmentation sphere in a two-dimensional or three-dimensional point of view by a user.

13. The three-dimensional virtual liver surgery planning system of claim 1, wherein
the liver segmentation module is configured to:
form a segment 1;
divide the liver into a left lobe and a right lobe;
divide the right lobe into an anterior sector and a posterior sector;
divide the left lobe into a medial sector (segment 4) and a lateral sector;
divide the posterior sector into a segment 6 and a segment 7;
divide the anterior sector into a segment 5 and a segment 8; and
divide the lateral sector into a segment 2 and a segment 3.

14. The three-dimensional virtual liver surgery planning system of claim 1, wherein
real-time calculated volume values of the extracted liver, vessels, tumors, and liver segments are represented.
